(19) United States
(12) Patent Application Publication
(10) Pub. No.: US 2017/0035991 A1
(43) Pub. Date: Feb. 9, 2017

(54) FORCE SENSING CATHETERS HAVING DEFLECTABLE STRUTS

(71) Applicant: Boston Scientific Scimed Inc., Maple Grove, MN (US)

(72) Inventors: Darrell L. Rankin, Milpitas, CA (US); John C. Potosky, San Jose, CA (US)

(21) Appl. No.: 15/230,285

(22) Filed: Aug. 5, 2016

Related U.S. Application Data
(60) Provisional application No. 62/202,334, filed on Aug. 7, 2015.

(51) Int. Cl. A61M 25/00; A61B 18/02; A61B 18/18; A61B 18/04

(52) U.S. Cl. CPC A61M 25/0097; A61B 18/04; A61B 18/02; A61B 18/18; A61M 2205/332; A61B 2018/00351

(57) ABSTRACT

Various embodiments concern a force sensing catheter having a proximal segment containing a proximal hub, a distal segment containing a distal hub, and a spring segment that extends from the proximal segment to the distal segment. The spring segment comprises a plurality of struts connected to both of the proximal and distal hubs. Each strut includes a preformed bend. The struts mechanically support the distal segment in a base orientation with respect to the proximal segment, flex at the preformed bends when the distal segment moves relative to the proximal segment in response to a force, and resiliently return the distal segment to the base orientation once the force has been removed. The catheter further comprises a plurality of sensors configured to output signals indicative of relative movement between the proximal and distal segments for determining a magnitude and direction of the force.

### CROSS-REFERENCE TO RELATED APPLICATION

This application claims priority to Provisional Application No. 62/202,334, filed Aug. 7, 2015, which is herein incorporated by reference in its entirety.

### TECHNICAL FIELD

The present disclosure relates generally to various force sensing catheter features.

### BACKGROUND

In ablation therapy, it may be useful to assess the contact between the ablation element and the tissue targeted for ablation. In interventional cardiac electrophysiology (EP) procedures, for example, the contact can be used to assess the effectiveness of the ablation therapy being delivered. Other catheter-based therapies and diagnostics can be aided by knowing whether a part of the catheter contacts targeted tissue, and to what degree the part of the catheter presses on the targeted tissue. The tissue exerts a force back on the catheter, which can be measured to assess the contact and the degree to which the catheter presses on the targeted tissue.

The present disclosure concerns, amongst other things, systems for measuring a force with a catheter.

### SUMMARY

The present disclosure relates to devices, systems, and methods for measuring a force experienced by a catheter.

In example 1, a catheter for measuring a force, the catheter comprising: a proximal segment containing a proximal hub; a distal segment containing a distal hub; a spring segment that extends from the proximal segment to the distal segment, the spring segment configured to permit relative movement between the distal segment and the proximal segment in response to application of the force on the distal segment, the spring segment comprising a plurality of struts, each strut comprising a preformed bend, wherein the plurality of struts are configured to: mechanically support the distal segment in a base orientation with respect to the proximal segment, flex at the preformed bends when the distal segment moves relative to the proximal segment in response to the force, and resiliently return the distal segment to the base orientation with respect to the proximal segment once the force has been removed; and a plurality of sensors configured to output a plurality of signals indicative of relative movement between the proximal and distal segments.

In example 2, the catheter of example 1, wherein the proximal hub is a proximal ring and the plurality of struts are respectively attached to the proximal ring at a plurality of proximal attachment points.

In example 3, the catheter of example 2, wherein the plurality of proximal attachment points are circumferentially arrayed around the proximal hub.

In example 4, the catheter of any of examples 1-3, wherein the distal hub is a distal ring and the plurality of struts are respectively attached to the distal ring at a plurality of distal attachment points.

In example 5, the catheter of example 4, wherein the plurality of distal attachment points are circumferentially arrayed around the distal hub.

In example 6, the catheter of any of examples 1-5, wherein: when the distal segment is in the base orientation with respect to the proximal segment, the proximal and distal hubs are coaxially aligned with a longitudinal axis and the plurality of struts are circumferentially arrayed around the longitudinal axis; and when the distal segment is moved out of the base orientation with respect to the proximal segment, the distal hub is no longer coaxially aligned with the longitudinal axis.

In example 7, the catheter of any of examples 1-6, wherein the preformed bends comprise respective curves having a bow profile.

In example 8, the catheter of example 7, wherein the bow profile of each of the curves is bowed radially inward with respect to the catheter.

In example 9, the catheter of any of examples 1-8, wherein the plurality of sensors are respectively mounted on the bends of the plurality of struts, and the signals output from the sensors respectively indicate changes in bending of the bends.

In example 10, the catheter of any of examples 1-9, wherein each of the plurality of struts comprises a first side and a second side opposite the first side, wherein the first side faces radially inward with respect to the catheter while the second side faces radially outward with respect to the catheter.

In example 11, the catheter of any of examples 1-10, wherein the plurality of sensors are respectively mounted on the first sides of the plurality of struts.

In example 12, the catheter of example 9, wherein the plurality of sensors are respectively mounted on the second sides of the plurality of struts.

In example 13, the catheter of any of examples 1-12, wherein the catheter further comprises a lumen that extends at least from the proximal segment to the distal segment, the lumen ending through the spring segment such that the plurality of struts are circumferentially arrayed around the lumen.

In example 14, the catheter of any of examples 1-14, wherein the plurality of sensors comprises a plurality of strain gauges.

In example 15, a system for measuring the force with the catheter of any preceding claim, the system comprising: the catheter; a user interface comprising a display; and control circuitry configured to: receive the plurality of signals; for each of the bends of the plurality of struts, determine an amount of strain that the bend experiences when the distal segment moves relative to the proximal segment based at least in part on the signal output from one of the plurality of sensors associated with the bend; based on the amount of strain determined for the bends of the plurality of struts, calculate a magnitude and a direction of the force; graphically indicate on the display the magnitude and the direction of the force.

In example 16, a catheter for measuring a force, the catheter comprising: a proximal segment containing a proximal hub; a distal segment containing a distal hub; a spring segment that extends from the proximal segment to the distal segment, the spring segment configured to permit relative movement between the distal segment and the proximal segment in response to application of the force on the distal segment, the spring segment comprising a plurality of struts, each strut comprising a proximal end attached to the proximal hub, a distal end attached to the distal hub, and a preformed bend located between the distal end and the proximal end, wherein the plurality of struts are configured to: mechanically support the distal segment in a base orientation with respect to the proximal segment, flex at the preformed bends when the distal segment moves relative to the proximal segment in response to the application of the force, and resiliently return the distal segment to the base orientation with respect to the proximal segment once the force has been removed; and a plurality of sensors configured to output a plurality of signals indicative of relative movement between the proximal and distal segments.

In example 17, the catheter of example 16, wherein the proximal hub is a proximal ring and the proximal ends of the plurality of struts are respectively attached to the proximal ring at a plurality of proximal attachment points.

In example 18, the catheter of example 17, wherein the plurality of proximal attachment points are circumferentially arrayed around the proximal hub.

In example 19, the catheter of any of examples 16-18, wherein the distal hub is a distal ring and the distal ends of the plurality of struts are respectively attached to the distal ring at a plurality of distal attachment points.

In example 20, the catheter of example 19, wherein the plurality of distal attachment points are circumferentially arrayed around the distal hub.

In example 21, the catheter of any of examples 16-20, wherein: when the distal segment is in the base orientation with respect to the proximal segment, the proximal and distal hubs are coaxially aligned with a longitudinal axis and the plurality of struts are circumferentially arrayed around the longitudinal axis; and when the distal segment is moved out of the base orientation with respect to the proximal segment, the distal hub is no longer coaxially aligned with the longitudinal axis.

In example, 22, the catheter of any of examples 16-21, wherein the preformed bends comprise respective curves having a bow profile.

In example 23, the catheter of example 22, wherein the bow profile of each of the curves is bowed radially inward with respect to the catheter.

In example 24, the catheter of any of example 16-23, wherein the plurality of sensors are respectively mounted on the bends of the plurality of struts, and the signals output from the sensors respectively indicate changes in bending of the bends.

In example 25, the catheter of any of example 16-24, wherein each of the plurality of struts comprises a first side and a second side opposite the first side, wherein the first side faces radially inward with respect to the catheter while the second side faces radially outward with respect to the catheter.

In example 26, the catheter of example 25, wherein the plurality of sensors are respectively mounted on the first sides of the plurality of struts.

In example 27, the catheter of example 25, wherein the plurality of sensors are respectively mounted on the second sides of the plurality of struts.

In example 28, the catheter of example 25, wherein for each strut of the plurality of struts, at least one sensor of the plurality of sensors is mounted on the first side of the strut and at least one other sensor of the plurality of sensors is mounted on the second side of the strut.

In example 29, the catheter of any of examples 16-28, further comprising a polymer tube having a lumen and a circumferential surface that defines an exterior of the catheter, wherein each of the proximal hub, the distal hub, and the plurality of struts are at least partially located within the lumen.

In example 30, the catheter of any of examples 16-29, wherein the catheter further comprises an ablation element located on the distal segment that is configured to deliver ablation therapy, and the control circuitry.

In example 31, the catheter of any of examples 16-30, wherein the catheter further comprises a lumen that extends at least from the proximal segment to the distal segment, the lumen ending through the spring segment such that the plurality of struts are circumferentially arrayed around the lumen.

In example 32, the catheter of any of examples 16-31, wherein the plurality of sensors comprises a plurality of strain gauges.

In example 33, a system for measuring the force with the catheter of any of claims 16-32, the system comprising: the catheter; a user interface comprising a display; and control circuitry configured to: receive the plurality of signals; for each of the bends of the plurality of struts, determine an amount of strain that the bend experiences when the distal segment moves relative to the proximal segment based at least in part on the signal output from one of the plurality of sensors associated with the bend; based on the amount of strain determined for the bends of the plurality of struts, calculate a magnitude and a direction of the force; graphically indicate on the display the magnitude and the direction of the force.

In example 34, a catheter for measuring a force, the catheter comprising: a proximal segment; a distal segment; a spring segment that extends from the proximal segment to the distal segment, the spring segment configured to permit relative movement between the distal segment and the proximal segment in response to application of the force on the distal segment, the spring segment comprising a plurality of struts, each strut extending from the proximal segment to the distal segment, each strut comprising a preformed curve that is bowed radially inward with respect to the catheter and each preformed curve is located between the distal segment and the proximal segment, wherein the plurality of struts are configured to: mechanically support the distal segment in a base orientation with respect to the proximal segment, flex at the preformed curves as the distal segment moves relative to the proximal segment in response to the application of the force, and resiliently return the distal segment to the base orientation with respect to the proximal segment once the force has been removed; and a plurality of sensors located on the bends of the struts and configured to output a plurality of signals indicative of dimensional changes in the curves of the struts.

In example 35, a system for measuring the force with the catheter of claim 1, the system comprising: a catheter comprising: a proximal segment containing a proximal hub; a distal segment containing a distal hub; a spring segment that extends from the proximal segment to the distal segment, the spring segment configured to permit relative movement between the distal segment and the proximal segment in response to application of the force on the distal segment, the spring segment comprising a plurality of struts, each strut comprising a proximal end attached to the proximal hub, a distal end attached to the distal hub, and a preformed curve located between the distal end and the proximal end, each curve bowed radially inward with respect to the catheter, wherein the plurality of struts are configured to: mechanically support the distal segment in a base orientation with respect to the proximal segment, flex at the preformed curves when the distal segment moves relative to the proximal segment in response to the application of the force, and resiliently return the distal segment to the base orientation with respect to the proximal segment once the force has been removed; and a plurality of sensors mounted on the curves and configured to output a plurality of signals indicative of dimensional changes in the curves of the struts; and control circuitry configured to: receive the plurality of signals; and calculate a magnitude and a direction of the force based on the plurality of signals.

While multiple embodiments are disclosed, still other embodiments of the present invention will become apparent to those skilled in the art from the following detailed description, which shows and describes various illustrative embodiments of the present disclosure. Accordingly, the drawings and detailed description are to be regarded as illustrative in nature and not restrictive.

### BRIEF DESCRIPTION OF THE DRAWINGS

FIGS. 1A-C show a system for measuring a force with a catheter in accordance with various embodiments of this disclosure.

FIG. 2 shows a block diagram of circuitry for controlling various functions described herein.

FIG. 3 shows a detailed perspective view of a distal end of a catheter in accordance with various embodiments of this disclosure.

FIG. 4 shows a perspective view of the inside of a catheter in accordance with various embodiments of this disclosure.

FIG. 5 shows a side view of the inside of a catheter in accordance with various embodiments of this disclosure.

FIG. 6 shows a cross sectional view taken along line AA of FIG. 5.

FIG. 7 shows a perspective view of hubs in accordance with various embodiments of this disclosure.

FIG. 8 shows an overhead view of a strut in accordance with various embodiments of this disclosure.

FIG. 9A-C shows a side view of a strut in different states of strain in accordance with various embodiments of this disclosure.

While the scope of the present disclosure is amenable to various modifications and alternative forms, specific embodiments have been shown by way of example in the drawings and are described in detail below. The intention, however, is not to limit the scope of the invention to particular embodiments described and/or shown. On the contrary, the invention is intended to cover all modifications, equivalents, and alternatives falling within the scope of the appended claims.

### DETAILED DESCRIPTION

Various cardiac abnormalities can be attributed to improper electrical activity of cardiac tissue. Such improper electrical activity can include, but is not limited to, generation of electrical signals, conduction of electrical signals, and/or mechanical contraction of the tissue in a manner that does not support efficient and/or effective cardiac function. For example, an area of cardiac tissue may become electrically active prematurely or otherwise out of synchrony during the cardiac cycle, thereby causing the cardiac cells of the area and/or adjacent areas to contract out of rhythm. The result is an abnormal cardiac contraction that is not timed for optimal cardiac output. In some cases, an area of cardiac tissue may provide a faulty electrical pathway (e.g., a short circuit) that causes an arrhythmia, such as atrial fibrillation or supraventricular tachycardia. In some cases, inactivate tissue (e.g., scar tissue) may be preferable to malfunctioning cardiac tissue.

Cardiac ablation is a procedure by which cardiac tissue is treated to inactivate the tissue. The tissue targeted for ablation may be associated with improper electrical activity, as described above. Cardiac ablation can lesion the tissue and prevent the tissue from improperly generating or conducting electrical signals. For example, a line, a circle, or other formation of lesioned cardiac tissue can block the propagation of errant electrical signals. In some cases, cardiac ablation is intended to cause the death of cardiac tissue and to have scar tissue reform over the lesion, where the scar tissue is not associated with the improper electrical activity. Lesioning therapies include electrical ablation, radiofrequency ablation, cyroablation, microwave ablation, laser ablation, and surgical ablation, among others. While cardiac ablation therapy is referenced herein as an exemplar, various embodiments of the present disclosure can be directed to ablation of other types of tissue and/or to non-ablation diagnostic and/or catheters that deliver other therapies.

Ideally, an ablation therapy can be delivered in a minimally invasive manner, such as with a catheter introduced to the heart through a vessel, rather than surgically opening the heart for direct access (e.g., as in a maze surgical procedure). For example, a single catheter can be used to perform an electrophysiology study of the inner surfaces of a heart to identify electrical activation patterns. From these patterns, a clinician can identify areas of inappropriate electrical activity and ablate cardiac tissue in a manner to kill or isolate the tissue associated with the inappropriate electrical activation. However, the lack of direct access in a catheter-based procedure may require that the clinician only interact with the cardiac tissue through a single catheter and keep track of all of the information that the catheter collects or is otherwise associated with the procedure. In particular, it can be challenging to determine the location of the therapy element (e.g., the proximity to tissue), the quality of a lesion, and whether the tissue is fully lesioned, under-lesioned (e.g., still capable of generating and/or conducting unwanted electrical signals), or over-lesioned (e.g., burning through or otherwise weakening the cardiac wall). The quality of the lesion can depend on the degree of contact between the ablation element and the targeted tissue. For example, an ablation element that is barely contacting tissue may not be adequately positioned to deliver effective ablation therapy. Conversely, an ablation element that is pressed too hard into tissue may deliver too much ablation energy or cause a perforation.

The present disclosure concerns, among other things, methods, devices, and systems for assessing a degree of contact between a part of a catheter (e.g., an ablation element) and tissue. Knowing the degree of contact, such as the magnitude and the direction of a force generated by contact between the catheter and the tissue, can be useful in determining the degree of lesioning of the targeted tissue. Information regarding the degree of lesioning of cardiac tissue can be used to determine whether the tissue should be further lesioned or whether the tissue was successfully ablated, among other things. Additionally or alternatively, an indicator of contact can be useful when navigating the catheter because a user may not feel a force being exerted on the catheter from tissue as the catheter is advanced within a patient, thereby causing vascular or cardiac tissue damage or perforation.

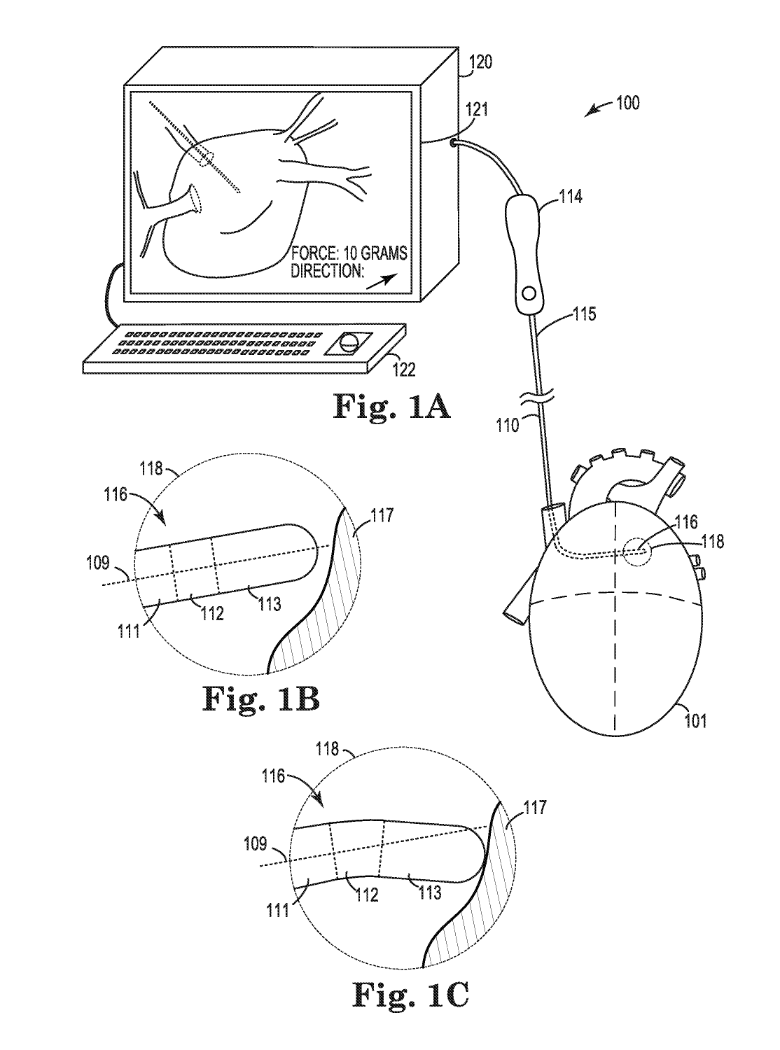

FIGS. 1A-1C illustrate an embodiment of a system 100 for sensing data from inside the body and/or delivering therapy. For example, the system 100 can be configured to map cardiac tissue and/or ablate the cardiac tissue, among other options. The system 100 includes a catheter 110 connected to a control unit 120 via handle 114. The catheter 110 can comprise an elongated tubular member having a proximal end 115 connected with the handle 114 and a distal end 116 configured to be introduced within a heart 101 or other area of the body. As shown in FIG. 1A, the distal end 116 of the catheter 110 is within the left atrium.

As shown in the window 118 of FIG. 1B, the distal end 116 of the catheter 110 includes a proximal segment 111, a spring segment 112, and a distal segment 113. The proximal segment 111, the spring segment 112, and the distal segment 113 can be coaxially aligned with each other in a base orientation as shown in FIG. 1B. Specifically, each of the proximal segment 111, the spring segment 112, and the distal segment 113 are coaxially aligned with a common longitudinal axis 109. The longitudinal axis 109 can extend through the radial center of each of the proximal segment 111, the spring segment 112, and the distal segment 113, and can extend through the radial center of the distal end 116 as a whole. The proximal segment 111, the spring segment 112, and the distal segment 113 can be mechanically biased to assume the base orientation. In some embodiments, the coaxial alignment of the proximal segment 111 with the distal segment 113 can correspond to the base orientation. As shown, the distal end 116, at least along the proximal segment 111, the spring segment 112, and the distal segment 113, extends straight. In some embodiments, this straight arrangement of the proximal segment 111, the spring segment 112, and the distal segment 113 can correspond to the base orientation.

The distal segment 113, or any other segment, can be in the form of an electrode configured for sensing electrical activity, such as electrical cardiac signals. Such an electrode (or other electrode on the catheter 110) can additionally or alternatively be used to deliver ablative energy to tissue.

The catheter 110 includes force sensing capabilities. For example, the catheter 110 is configured to sense a force due to engagement with tissue 117. The distal segment 113 can be relatively rigid while segments proximal of the distal segment 113 can be relatively flexible. In particular, the spring segment 112 may be more flexible than the distal segment 113 and the proximal segment 111 such that when the distal end 116 of the catheter 110 engages tissue 117, the spring segment 112, as shown in FIG. 1C, bends. For example, the distal end 116 of the catheter 110 can be generally straight as shown in FIG. 1B. When the distal segment 113 engages tissue 117, the distal end 116 of the catheter 110 can bend at the spring segment 112 such that the distal segment 113 moves relative to the proximal segment 111. As shown in FIGS. 1B and 1C, the normal force from the tissue moves the distal segment 113 out of coaxial alignment (e.g., with respect to the longitudinal axis 109) with the proximal segment 111 while the spring segment 112 bends. As such, proximal segment 111 and the distal segment 113 may be stiff to not bend due to the force while the spring segment 112 may be less stiff and bend to accommodate the force exerted on the distal end 116 of the catheter 110. One or more sensors within the distal end 116 of the catheter 110 can sense the degree of bending of the spring segment 112 to determine the magnitude and the direction of the force, as further discussed herein.

The control unit 120 of the system 100 includes a display 121 (e.g., LCD) for displaying information. The control unit 120 further includes a user input 122 which can comprise one or more buttons, toggles, a track ball, a mouse, touchpad, or the like for receiving user input. The user input 122 can additionally or alternatively be located on the handle 114. The control unit 120 can contain control circuitry for performing the functions referenced herein. Some or all of the control circuitry can alternatively be located within the handle 114.

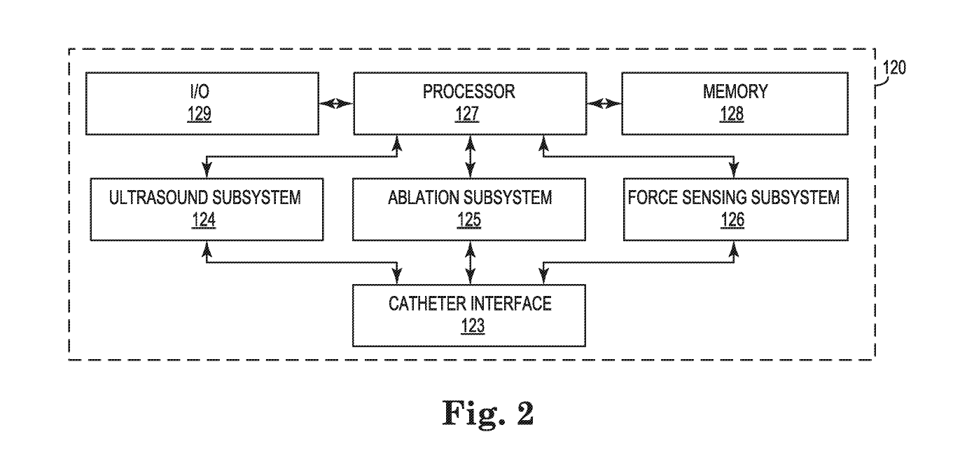

FIG. 2 illustrates a block diagram showing an example of control circuitry which can perform functions referenced herein. This or other control circuitry can be housed within control unit 120, which can comprise a single housing or multiple housings among which components are distributed. Control circuitry can additionally or alternatively be housed within the handle 114. The components of the control unit 120 can be powered by a power supply (not shown), known in the art, which can supply electrical power to any of the components of the control unit 120 and the system 100. The power supply can plug into an electrical outlet and/or provide power from a battery, among other options.

The control unit 120 can include a catheter interface 123. The catheter interface 123 can include a plug which receives a cord from the handle 114. The catheter 110 can include multiple conductors (not illustrated but known in the art) to convey electrical signals between the distal end 116 and the proximal end 115 and further to the catheter interface 123. It is through the catheter interface 123 that the control unit 120 (and/or the handle 114 if control circuitry is included in the handle 114) can send electrical signals to any element within the catheter 110 and/or receive an electrical signal from any element within the catheter 110. The catheter interface 123 can conduct signals to any of the components of the control unit 120.

The control unit 120 can include an ultrasound subsystem 124 which includes components for operating the ultrasound functions of the system 100. While the illustrated example of control circuitry shown in FIG. 2 includes the ultrasound subsystem 124, it will be understood that not all embodiment may include ultrasound subsystem 124 or any circuitry for imaging tissue. The ultrasound subsystem 124 can include a signal generator configured to generate a signal for ultrasound transmission and signal processing components (e.g., a high pass filter) configured to filter and process reflected ultrasound signals as received by an ultrasound transducer in a sense mode and conducted to the ultrasound subsystem 124 through a conductor in the catheter 110. The ultrasound subsystem 124 can send signals to elements within the catheter 110 via the catheter interface 123 and/or receive signals from elements within the catheter 110 via the catheter interface 123.

The control unit 120 can include an ablation subsystem 125. The ablation subsystem 125 can include components for operating the ablation functions of the system 100. While the illustrated example of control circuitry shown in FIG. 2 includes the ablation subsystem, it will be understood that not all embodiment may include ablation subsystem 125 or any circuitry for generating an ablation therapy. The ablation subsystem 125 can include an ablation generator to provide different therapeutic outputs depending on the particular configuration (e.g., a high frequency alternating current signal in the case of radiofrequency ablation to be output through one or more electrodes). Providing ablation energy to target sites is further described, for example, in U.S. Pat. No. 5,383,874 and U.S. Pat. No. 7,720,420, each of which is expressly incorporated herein by reference in its entirety for all purposes. The ablation subsystem 125 may support any other type of ablation therapy, such as microwave ablation. The ablation subsystem 125 can deliver signals or other type of ablation energy through the catheter interface 123 to the catheter 110.

The control unit 120 can include a force sensing subsystem 126. The force sensing subsystem 126 can include components for measuring a force experienced by the catheter 110. Such components can include signal processors, analog-to-digital converters, operational amplifiers, comparators, and/or any other circuitry for conditioning and measuring one or more signals. The force sensing subsystem 126 can send signals to elements within the catheter 110 via the catheter interface 123 and/or receive signals from elements within the catheter 110 via the catheter interface 123.

Each of the ultrasound subsystem 124, the ablation subsystem 125, and the force sensing subsystem 126 can send signals to, and receive signals from, the processor 127. The processor 127 can be any type of processor for executing computer functions. For example, the processor 127 can execute program instructions stored within the memory 128 to carry out any function referenced herein, such as determine the magnitude and direction of a force experienced by the catheter 110.

The control unit 120 further includes an input/output subsystem 129 which can support user input and output functionality. For example, the input/output subsystem 129 may support the display 121 to display any information referenced herein, such as a graphic representation of tissue, the catheter 110, and a magnitude and direction of the force experienced by the catheter 110, amongst other options. Input/output subsystem 129 can log key and/or other input entries via the user input 122 and route the entries to other circuitry.

A single processor 127, or multiple processors, can perform the functions of one or more subsystems, and as such the subsystems may share control circuitry. Although different subsystems are presented herein, circuitry may be divided between a greater or lesser numbers of subsystems, which may be housed separately or together. In various embodiments, circuitry is not distributed between subsystems, but rather is provided as a unified computing system. Whether distributed or unified, the components can be electrically connected to coordinate and share resources to carry out functions.

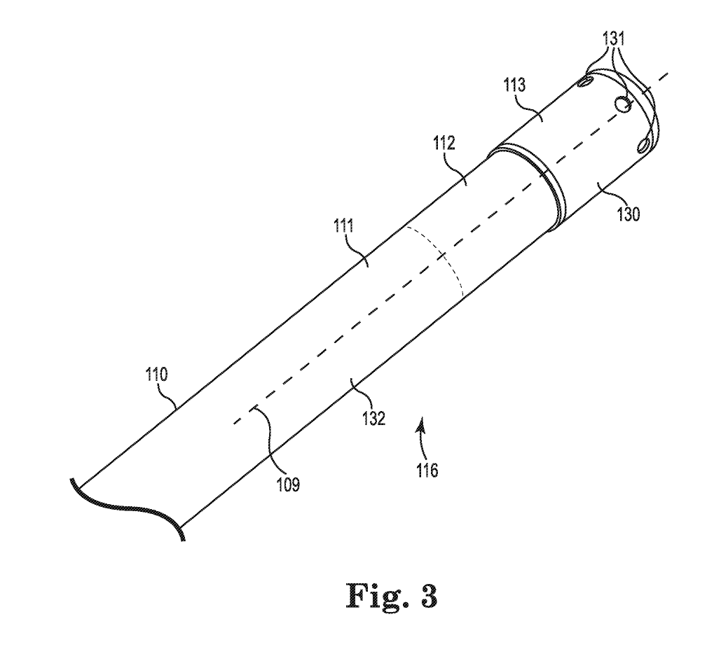

FIG. 3 illustrates a detailed view of the distal end 116 of the catheter 110. FIG. 3 shows a catheter shaft 132. The catheter shaft 132 can extend from the distal segment 113 to the handle 114 (FIG. 1A), and thus can define an exterior surface of the catheter 110 along the spring segment 112, the proximal segment 111, and further proximally to the proximal end 115 (FIG. 1A). The catheter shaft 132 can be a polymeric tube formed from various polymers, such as polyurethane, polyamide, polyether block amide, silicone, and/or other materials. In some embodiments, the catheter shaft 132 may be relatively flexible, and at least along the spring segment 112 may not provide any material mechanical support to the distal segment 113 (e.g., facilitated by thinning of the wall of the catheter shaft 132 along the spring segment 112).

As shown, the proximal segment 111 can be proximal and adjacent to the spring segment 112. The length of the proximal segment 111 can vary between different embodiments, and can be five millimeters to five centimeters, although different lengths are also possible. The length of the spring segment 112 can also vary between different embodiments and is dependent on the length of underlying struts as will be further discussed herein. The spring segment 112 is adjacent to the distal segment 113. As shown in FIG. 3, the distal segment 113 can be defined by an electrode 130. The electrode 130 can be an ablation electrode. In some other embodiments, the distal segment 130 may not be an electrode. The electrode 130 can be in a shell form which can contain other components. The electrode 130 can include a plurality of ports 131. One or more ultrasonic transducers, housed within the electrode 130, can transmit and receive signals through the ports 131 or through additional dedicated holes in the tip shell. Additionally or in place of the transducers, one or more miniature electrodes may be incorporated into the tip shell assembly.

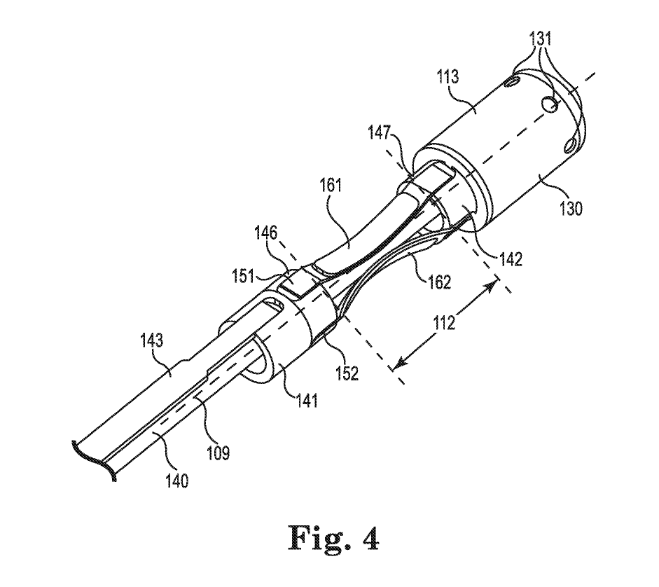

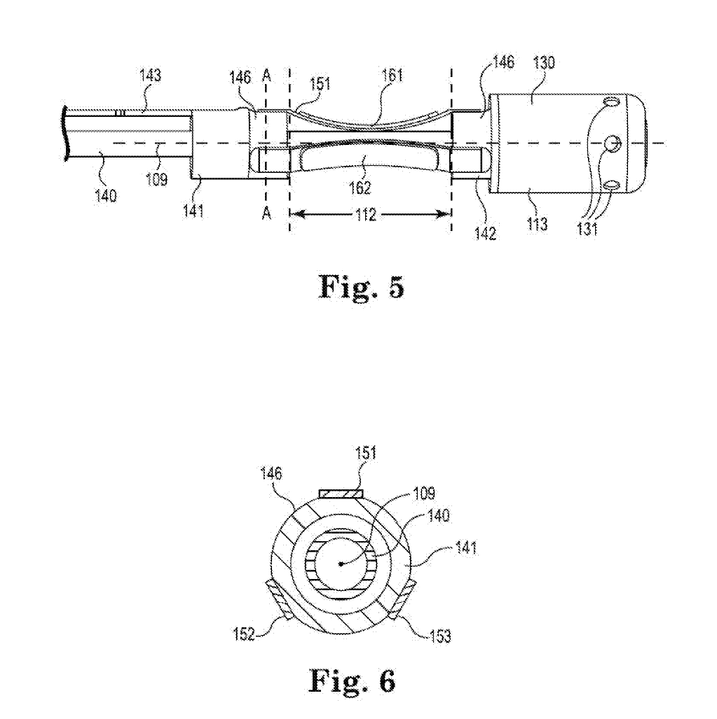

FIG. 4 shows distal end 116 of the catheter 110 of FIG. 3 after the removal of the catheter shaft 132 to expose various components that underlie the catheter shaft 132. FIG. 5 shows a side view of the distal end 116 of the catheter 110 of FIG. 3 with the shaft 132 removed, as with FIG. 4. The removal of the catheter shaft 132 exposes structural and force sensing components. The force sensing components can include a proximal hub 141, a distal hub 142, and a plurality of struts 151-153 (strut 153 shown in FIG. 6) that bridge between the proximal hub 141 and the distal hub 142. The proximal hub 141 and the distal hub 142 can be respective rings to which the plurality of struts 151-153 are attached. One or both of the proximal hub 141 and the distal hub 142 can be formed from polymer materials, such as polyethylene, PEEK or can be formed from a metal, such as stainless steel. One or both of the proximal hub 141 and the distal hub 142 can be formed from a composite of metal, polymer, and/or other materials.

The proximal hub 141 and the distal hub 142 can be coaxially aligned with respect to the longitudinal axis 109. For example, the longitudinal axis 109 can extend through the respective radial centers of each of the proximal hub 141 and the distal hub 142. An inner tube 140 can extend through the catheter 110 (e.g., to the handle 114, FIG. 1A), through the proximal hub 141 and the distal hub 142. The inner tube 140 can include one or more lumens within which one or more conductors (not illustrated) can extend from the proximal end 115 (FIG. 1A) to the distal segment 113, such as for connecting with one or more electrical elements (e.g., ultrasound transducer, electrode, stain sensor, or other component). Coolant fluid can additionally or alternatively be routed through the inner tube 140. In various embodiments, the catheter 110 is open irrigated (e.g., through the plurality of ports 131) to allow the coolant fluid to flow out of the distal segment 113. Various other embodiments concern a non-irrigated catheter 110.

A tether 143 can attach to a proximal end of the proximal hub 141. Considering FIGS. 1A, 4, and 5 together, the tether 143 can attach to a deflection mechanism within the handle 114 to cause deflection of the distal end 116. A knob, slider, or plunger on a handle 114 may be used to create tension or slack in the tether 143.

As shown in FIGS. 4 and 5, the spring segment 112 can extend from a distal edge of the proximal hub 141 to a proximal edge of the distal hub 142. As such, the proximal hub 141 can be part of, and may even define the length of, the proximal segment 111 (FIG. 1A). Likewise, the distal hub 142 can be part of the distal segment 113. The spring segment 112 can be a relatively flexible portion that is mostly or entirely mechanically supported by the plurality of struts 151-153. As such, the proximal hub 141 and the distal hub 142 can be stiffer than the plurality of struts 151-153 such that a force directed on the distal segment 113 causes the distal end 116 to bend along the plurality of struts 151-153 (the spring segment 112 specifically) rather than along the distal segment 113 or the proximal segment 111.

The proximal hub 141 includes an attachment portion 146. The attachment portion 146 can be on a distal side of the proximal hub 141. Proximal ends of the plurality of struts 151-153 can be attached to the attachment portion 146. The distal hub 142 can include an attachment portion 147. The attachment portion 147 can be on a proximal side of the distal hub 142. Distal ends of the plurality of struts 151-153 can be attached to the attachment portion 147. The length of the spring segment 112 may be defined as the length of the plurality of struts 151-153 that is not overlapped by either of the proximal hub 141 or the distal hub 142 because this is the portion of the distal end 116 which is configured to bend due to a force.

The plurality of struts 151-153 are circumferentially arrayed around the longitudinal axis 109 such that one or more of the struts will be compressed when the distal segment 113 moves relative to the proximal segment 111 while one or more of the other struts will be stretched when the distal segment 113 moves relative to the proximal segment 111. Which struts elongate or compress depends on the direction of the force. If the force had a different direction, a different one or more of the struts will be compressed while a different one or more of the struts will be stretched. Based on the different amounts of stretching and compressing of the struts 151-153, and which struts 151-153 compress and which struts 151-153 elongate, the magnitude and direction of force can be determined by the force sensing subsystem 126. In particular, a plurality of strain sensors 161-163 (one of the strain sensors 163 being located on the strut 153 hidden from view in FIGS. 4 and 5) can sense the compression or strain in the plurality of struts 151-153 to determine the magnitude and direction of the force. The plurality of strain sensors 161-163 can be any type of strain sensor, including electro-resistive (e.g., an electrical conductor that changes in resistivity based on strain) or optical (an optical characteristic of a light conducting element that changes as the element is strained).

Strain sensors 161-163 may comprise metallic foil gauges or semiconductor gauges which indicate dimensional changes (e.g., elongation, compaction) by a change in resistance of a metal or a semiconductor. Strain sensors 161-163 may comprise piezoelectric crystals which output current based on strain. Strain sensors 161-163 may comprise a fiber optic element through which radiation is propagated. The fiber optic element may change in some optical characteristic, such as resonant wavelength of a diffraction grating, based on strain of the fiber optic element. It will be understood that electrode conductors, fiber optic elements, and/or other signal carrying elements, while not shown but are known, can extend from the strain sensors 161-163 proximally to convey signals between the strain sensors 161-163 and the force sensing subsystem 126. Such signal carrying elements can extend within the catheter shaft 132 (FIG. 3) and optionally further within the inner tube 140.

FIG. 6 shows a cross-sectional view along line AA of FIG. 5. In particular, the cross-sectional view cuts through the proximal hub 141. All three struts 151-153 are shown in FIG. 6. As shown, the struts 151-153 are circumferentially arrayed around the proximal hub 141 (and likewise can be circumferentially arrayed around the distal hub 142 in the same manner), the inner tube 140, and the longitudinal axis 109. The respective centers of the three struts 151-153 can be separated by 120 degrees, for example. It will be understood that a different number of struts can alternatively be provided, such as two, four, five, or more. The struts can be evenly spaced circumferentially around the proximal hub 141 (and likewise around the distal hub 142 in the same manner), the inner tube 140, and/or the longitudinal axis 109. Any number of strain sensors, optionally equal to the number of struts, can be located on the struts (e.g., one strain sensor mounted on each strut or two strain sensors mounted on each strut).

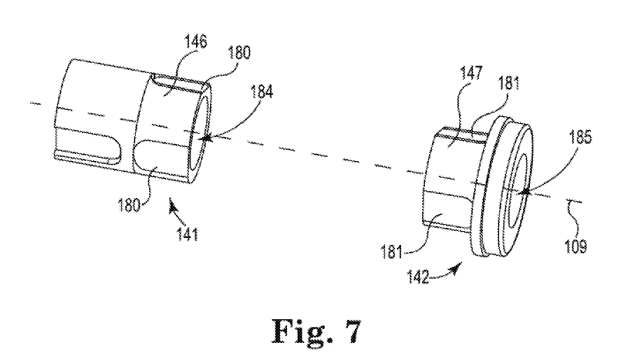

FIG. 7 shows perspective views of the proximal hub 141 and the distal hub 142 in respective isolation. As such, the proximal hub 141 includes a lumen 184 and the distal hub 142 includes a lumen 185. Conductors, the inner tube 140 or other elements can extend through the lumens 184, 185. The proximal hub 141 includes a plurality of attachment surfaces 180. As shown, each attachment surface 180 can be flat while the rest of the attachment portion 146 is relatively round. As such, the attachment portion 146 can comprise alternating flat and round section that extends around the circumference of the proximal hub 141. Each attachment surface 180 can serve as a surface to interface with a flat, proximal portion of a respective one of the struts 151-153. The struts 151-153 can be attached to the attachment portion 146 at such attachment surfaces 180. The struts 151-153 can be attached to the proximal hub 141 by an adhesive (e.g., epoxy), welding, and/or riveting. In some embodiments, a collar may be placed over the proximal ends of the struts 151-153 to pinch the proximal ends of the struts 151-153 between the collar and the proximal hub 141 to attach the struts 151-153 to the proximal hub 141.

The distal hub 142 includes a plurality of attachment surfaces 181. Each attachment surface 181 can be flat while the rest of the attachment portion 147 can be relatively round. As such, the attachment portion 147 can comprise alternating flat and round section that extends around the circumference of the distal hub 142. Each attachment surface 181 can serve as a surface to interface with a flat, distal portion of a respective one of the struts 151-153. The struts 151-153 can be attached to the attachment portion 147 at such attachment surfaces 181. The struts 151-153 can be attached to the distal hub 142 by an adhesive (e.g., epoxy), welding, and/or riveting. In some embodiments, a collar may be placed over the distal ends of the struts 151-153 to pinch the distal ends of the struts 151-153 between the collar and the distal hub 142 to attach the struts 151-153 to the distal hub 142.

The struts can be circumferentially arrayed around each of the proximal hub 141 and the distal hub 142. The circumference (or diameter) of the attachment portion 146 of the proximal hub 141 can be equal to the circumference (or diameter) of the attachment portion 147 of the distal hub 142. The attachment of the struts 151-153 to the proximal hub 141 and the distal hub 142 can secure the distal hub 142 to the proximal of 141 while allowing movement of the distal hub 142 relative to the proximal hub 141. Furthermore, the struts 151-153 can be structurally resilient to return the distal hub 142 back to the base orientation (e.g., coaxial with longitudinal axis 109) with respect to the proximal hub 141 once an external force to the catheter has been removed.

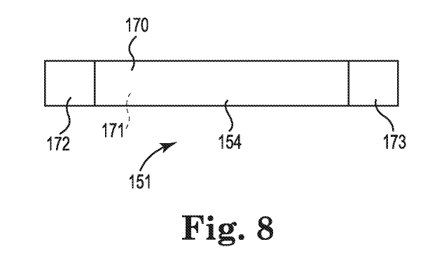

FIG. 8 shows an overhead view of strut 151 in isolation. Being that the struts 151-153 can be identical, the view of strut 151, and the discussion herein, can apply to any of the struts. As shown in the overhead view of FIG. 8, the strut has a proximal portion 172, a distal portion 173, and a bend 154 which extends from the proximal portion 172 to the distal portion 173. As shown in the view of FIG. 8, strut 151 has the profile of a rectangular strip. The strut 151 includes the first side 171 and a second side 170 opposite the first side 171. The first side can extend over each of the proximal portion 172, the bend 170, and the distal portion 173. Likewise the second side 170 can extend over each of the proximal portion 172, the bend 170, and the distal portion 173.

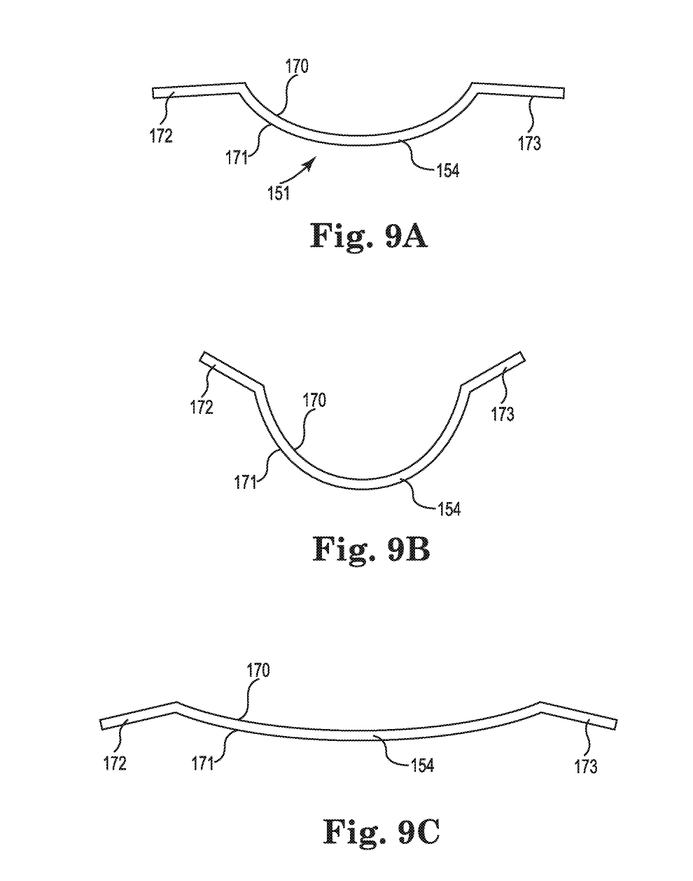

FIGS. 9A-C show isolated views of different states of the strut 151. While strut 151 is shown, FIGS. 9A-C and associated discussion can represent the mechanics of any strut referenced herein. The strut 151 includes a proximal portion 172, a bend 154, and a distal portion 173. The proximal portion 172 can be flat, the distal portion 173 can be flat, and the bend 154 can be in a nonplanar configuration. The bend 154 of the strut 151 can extend proximally to the proximal portion 172 and distally to the distal portion 173. For example, the proximal portion 172 can be coplanar with the distal portion 173, while the bend 154 can be curved therebetween.

The strut 151 can be a unitary piece of metal, such as a nickel titanium alloy (nitinol), stainless steel, aluminum, titanium, or other metal. The strut 151 can alternatively be made from polymers, such as polyimide or polyethylene. The strut 151 can be a composite, such as by being made of a metal and a polymer. Alternately the entire spring segment 112, containing a plurality of struts 151-153 and hubs 141-142 could be formed from a tube segment that is laser cut or machined to the desired strut profiles and characteristics. It is noted that in some embodiments, one or more signals may be conducted through the struts 151-153, respectively. For example, an RF signal may be conducted to the electrode 130 (FIGS. 4 and 5) through one or all of the struts 151-153. In such embodiments, a conductor may be attached to the proximal ends of the struts 151-153.

Considering FIGS. 7 and 9A-9C together, the proximal portion 172 and the distal portion 173 can be shaped to interface with the attachment surfaces 180, 181 of the proximal hub 141 and the distal hub 142, respectively, for attachment therebetween. The proximal portion 172 can contact, and be directly attached to, the attachment portion 146 (e.g., a flat portion of the attachment portion 146). For example, the proximal portion 172 can be adhered with an adhesive, can be welded, or can be riveted, amongst other options, to the proximal hub 141 (e.g., to attachment surface 181 of the attachment portion 146). The distal portion 173 can contact, and be directly attached to, the attachment portion 147 (e.g., a flat portion of the attachment portion 147). For example, the distal portion 173 can be adhered with an adhesive, can be welded, or can be riveted, amongst other options, to the distal hub 142 (e.g., to attachment surface 182 of the attachment portion 147).

It is noted that the first side 171 is radially inward facing while the second side 170 is radially outward facing in FIGS. 4 and 5. In this way, the struts 151-153 bow radially inward. The bowing of the bend 154 radially inward means that the strut 151 will further bow inward when compressed, thereby keeping the profile of the assembly compact. The inner tube 140 or other element may serve to bottom out the bowing of the struts 151-153 (e.g., by contact between the bends of the struts and the inner tube 140 or other element) to prevent potentially damaging over-compression. The struts 151-153 may alternatively bow radially outward, however bending of the struts 151-153 outward increases the overall radius of the array of struts 151-153 thereby increasing the hoop strength of the array of struts 151-153. Being that it may not be desirable for the array of struts 151-153 to increasing in strength when attempting to measure a force, it may be preferable to have the pre-formed bends to bow radially inward rather than outward.

A strain sensor 161-163 can be placed on one or both of the first side 171 or the second side 170 (e.g., as shown in FIGS. 4 and 5). If a pair of strain sensors are located on each strut (e.g., located on the first side 171 and the second side 170 respectively), the sensors may be electrically connected to a differential amplifier to determine the difference between the strain in the opposite sides of the strut. Such a differential signal may be used to determine the overall compression or elongation of the bend of a strut and cancel out temperature or other unwanted effects.

FIG. 9A shows the strut 151 in an unstrained state. For example, the strut 151 can be pre-biased to assume the shape shown in FIG. 9A. FIG. 9B shows the strut 151 in a stretched state. FIG. 9C shows the strut 151 in a compressed state. If the strut 151 is placed in either of the stretched state or compressed state by the force placed on the catheter 110, the strut 151 will resiliently return to the pre-biased state shown in FIG. 9A once the force is removed. As such, the plurality of struts 151-153 can structurally support the distal segment 113 from the proximal segment 111, can allow the distal segment 113 to move relative to the proximal segment 111 based on a force exerted on the distal segment 113, and can resiliently return the distal segment 113 to its original orientation with respect to the proximal segment 111 once the force has been removed. It is noted that the plurality of struts 151-153 may provide most or all of the mechanical support that holds the distal segment 113 in the base orientation with respect to the proximal segment 111 and resiliently returns the distal segment 113 to the base orientation with respect to the proximal segment 111 after remove of a force. The compression and elongation of the struts 151-153 during such relative movement of the distal segment 113 and the proximal segment 111 can be measured to determine the magnitude of the force and the direction force, as further discussed herein.

If the force exerted on the distal segment 113 is coaxial with the longitudinal axis 109, then each of the struts 151-153 will compress in equal amounts. The plurality of strain sensors 161-163 will measure these equal amounts of dimensional change in the bends of the struts 151-153. Based on these equal changes, the control circuitry can determine a magnitude and direction of the force. The magnitude of the force can be calculated using Hooke's law, wherein the displacement of a spring element (e.g., strut 151) is proportional to the forced placed on element, based on a predetermined constant. Being that the displacements are equal for each of the struts 151-153, the control circuitry can determine that the force is coaxial with the longitudinal axis 109. If the force is not coaxial with the longitudinal axis 109, then one or more of the struts will be in compression (e.g., by as shown in FIG. 9B) while one or more of the struts are in tension (e.g., as shown in FIG. 9C) relative to the state shown in FIG. 9A. The distal segment 113 will tend to curl or shift radially away from the force with respect to the proximal segment 111. Therefore, the one or more struts in tension indicate the direction from which the force is coming while the one or more struts in compression indicate the opposite direction (in which the force is pointed or going). Based on this, the direction (e.g., unit vector) of the force can be determined by the control circuitry.

The pre-bending of the strut 151 ensures that the bend 154 will experience much if not all of the overall bending of the strut 151. Therefore, locating the strain sensor 161 along each bend ensures that the strain sensor 161 will capture most if not all of the elongation or compaction of the strut 151. This increases the sensitivity of the system by focusing the bending at the location of the strain sensor 161. The location of strain sensor 161 can be limited to the bend 154 and may not be located distally, proximally, or laterally from the bend 154 portion of the strut 151 (e.g., may not be on the proximal portion 172 or the distal portion 173). The struts can also undergo a torsional response to applied for in addition to just tension or compression. Additional strain sensors can be specifically oriented and or layered to capture this motion and provide additional signal generation.

Once assembled, the catheter 110 may undergo a calibration step, either at a factory or just before use by a physician. In such a step, a plurality of forces of known magnitude and direction can be placed, in sequence, on the distal segment 113 to move the distal segment 113 relative to the proximal segment 111 while the strain sensors 161-163 output signals indicative of the bending of the struts 151-153. A table can be generated indicating a separate entry for each force. Thereafter, a force of unknown magnitude and/or direction can be analyzed by comparing signals output from the strain sensors 161-163 to the values of the table to identify the best match. Specifically, in the case of electro-resistive strain sensors, such an algorithm can identify which entry from the calibration data has three (or other number depending on the number of strain sensors) change-in-resistance values best matching the current change-in-resistance values. The magnitude and direction of the known force from the calibration step can be indicated as the magnitude and direction currently being experienced. In some cases, a mathematical relationship can be generated based on the linearity of Hooke's law, wherein a limited number of calibration steps are performed to determine the change-in-resistance, or other output from the strain sensors, and interpolation and/or extrapolation can be computed based on these calibration values. For example, the spring constant can be determined for a strut such that a subsequent elongation or contraction amount, as determined by strain sensor, can be multiplied by the spring constant to determine the magnitude of the force acting on the distal segment 113 (and thus the strut). The deflection of multiple struts can be factored for determining an overall magnitude and direction for the force.

In some embodiments, the magnitude and direction of the force that are indicated to the user indicates the magnitude and the direction of a force that acts on the distal segment 113. This force typically results from the distal segment 113 pushing against tissue. Therefore, the force acting on the distal segment 113 may be a normal force resulting from the force that the distal segment 113 exerts on the tissue. In some embodiments, it is the force acting on the distal segment 113 that is calculated and represented to a user. Additionally or alternatively, it is the force that the distal segment 113 applies to tissue that is calculated and represented to the user.

The magnitude can be represented in grams or another measure of force. The magnitude can be presented as a running line graph that moves over time to show new and recent force values. The direction can be represented as a unit vector in a three dimensional reference frame (e.g., relative to a X, Y, and Z axes coordinate system). In some embodiments, a three dimensional mapping function can be used to track the three dimensional position of the distal end 116 of the catheter 110 in the three dimensional reference frame. Magnetic fields can be created outside of the patient and sensed by a sensor that is sensitive to magnetic fields within distal end 116 of the catheter 110 to determine the three dimensional position of the distal end 116 of the catheter 110 in the three dimensional reference frame. The direction can be represented relative to the distal end 116 of the catheter 110. For example, a line projecting to, or from, the distal segment 113 can represent the direction of the force relative to the distal segment 113. Such representations can be made on a display as discussed herein.

The magnitude and direction of the force can be used for navigation by providing an indicator when the catheter encounters tissue and/or for assessing the lesioning of tissue by determining the degree of contact between the lesioning element and the tissue, among other options. In some embodiments, a force under 10 grams is suboptimal for lesioning tissue (e.g., by being too small) while a force over 40 grams is likewise suboptimal for lesioning tissue (e.g., by being too large). Therefore, a window between 10 and 40 grams may be ideal for lesioning tissue and the output of the force during lesioning may provide feedback to the user to allow the user to stay within this window. Of course, other force ranges ideal for lesioning may be used.

The techniques described in this disclosure, including those attributed to a system, control unit, control circuitry, processor, or various constituent components, may be implemented wholly or at least in part, in hardware, software, firmware or any combination thereof. A processor, as used herein, refers to any number and/or combination of a microprocessor, a digital signal processor (DSP), an application specific integrated circuit (ASIC), a field-programmable gate array (FPGA), microcontroller, discrete logic circuitry, processing chip, gate arrays, and/or any other equivalent integrated or discrete logic circuitry. As part of control circuitry, at least one of the foregoing logic circuitry can be used, alone or in combination with other circuitry, such as memory or other physical medium for storing instructions, can be used to carry about specified functions (e.g., a processor and memory having stored program instructions executable by the processor for determining a magnitude and a direction of a force exerted on a catheter). The functions referenced herein may be embodied as firmware, hardware, software or any combination thereof as part of control circuitry specifically configured (e.g., with programming) to carry out those functions, such as in means for performing the functions referenced herein. The steps described herein may be performed by a single processing component or multiple processing components, the latter of which may be distributed amongst different coordinating devices. In this way, control circuitry may be distributed between multiple devices. In addition, any of the described units, modules, subsystems, or components may be implemented together or separately as discrete but interoperable logic devices of control circuitry. Depiction of different features as modules, subsystems, or units is intended to highlight different functional aspects and does not necessarily imply that such modules or units must be realized as hardware or software components and/or by a single device. Rather, specified functionality associated with one or more module, subsystem, or units, as part of control circuitry, may be performed by separate hardware or software components, or integrated within common or separate hardware or software components of control circuitry.

When implemented in software, the functionality ascribed to the systems, devices, and control circuitry described in this disclosure may be embodied as instructions on a physically embodied computer-readable medium such as RAM, ROM, NVRAM, EEPROM, FLASH memory, magnetic data storage media, optical data storage media, or the like, the medium being physically embodied in that it is not a carrier wave, as part of control circuitry. The instructions may be executed to support one or more aspects of the functionality described in this disclosure.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as falling within the scope of the claims, together with all equivalents thereof.

What is claimed is:

1. A catheter for measuring a force, the catheter comprising:
a proximal segment containing a proximal hub;
a distal segment containing a distal hub;
a spring segment that extends from the proximal segment to the distal segment, the spring segment configured to permit relative movement between the distal segment and the proximal segment in response to application of the force on the distal segment, the spring segment comprising a plurality of struts, each strut comprising a proximal end attached to the proximal hub, a distal end attached to the distal hub, and a preformed bend located between the distal end and the proximal end, wherein the plurality of struts are configured to:
mechanically support the distal segment in a base orientation with respect to the proximal segment,
flex at the preformed bends when the distal segment moves relative to the proximal segment in response to the application of the force, and
resiliently return the distal segment to the base orientation with respect to the proximal segment once the force has been removed; and
a plurality of sensors configured to output a plurality of signals indicative of relative movement between the proximal and distal segments.

2. The catheter of claim 1, wherein the proximal hub is a proximal ring and the proximal ends of the plurality of struts are respectively attached to the proximal ring at a plurality of proximal attachment points

3. The catheter of claim 2, wherein the plurality of proximal attachment points are circumferentially arrayed around the proximal hub.

4. The catheter of claim 1, wherein the distal hub is a distal ring and the distal ends of the plurality of struts are respectively attached to the distal ring at a plurality of distal attachment points.

5. The catheter of claim 4, wherein the plurality of distal attachment points are circumferentially arrayed around the distal hub.

6. The catheter of claim 1, wherein:
when the distal segment is in the base orientation with respect to the proximal segment, the proximal and distal hubs are coaxially aligned with a longitudinal axis and the plurality of struts are circumferentially arrayed around the longitudinal axis; and
when the distal segment is moved out of the base orientation with respect to the proximal segment, the distal hub is no longer coaxially aligned with the longitudinal axis.

7. The catheter of claim 1, wherein the preformed bends comprise respective curves having a bow profile.

8. The catheter of claim 7, wherein the bow profile of each of the curves is bowed radially inward with respect to the catheter.

9. The catheter of claim 1, wherein the plurality of sensors are respectively mounted on the bends of the plurality of struts, and the signals output from the sensors respectively indicate changes in bending of the bends.

10. The catheter of claim 1, wherein each of the plurality of struts comprises a first side and a second side opposite the first side, wherein the first side faces radially inward with respect to the catheter while the second side faces radially outward with respect to the catheter.

11. The catheter of claim 10, wherein the plurality of sensors are respectively mounted on the first sides of the plurality of struts.

12. The catheter of claim 10, wherein the plurality of sensors are respectively mounted on the second sides of the plurality of struts.

13. The catheter of claim 10, wherein for each strut of the plurality of struts, at least one sensor of the plurality of sensors is mounted on the first side of the strut and at least one other sensor of the plurality of sensors is mounted on the second side of the strut.

14. The catheter of claim 1, further comprising a polymer tube having a lumen and a circumferential surface that defines an exterior of the catheter, wherein each of the proximal hub, the distal hub, and the plurality of struts are at least partially located within the lumen.

15. The catheter of claim 1, wherein the catheter further comprises an ablation element located on the distal segment that is configured to deliver ablation therapy, and the control circuitry

16. The catheter of claim 1, wherein the catheter further comprises a lumen that extends at least from the proximal segment to the distal segment, the lumen ending through the spring segment such that the plurality of struts are circumferentially arrayed around the lumen.

17. The catheter claim 1, wherein the plurality of sensors comprises a plurality of strain gauges.

18. A system for measuring the force with the catheter of claim 1, the system comprising:
the catheter;
a user interface comprising a display; and
control circuitry configured to:
receive the plurality of signals;
for each of the bends of the plurality of struts, determine an amount of strain that the bend experiences when the distal segment moves relative to the proximal segment based at least in part on the signal output from one of the plurality of sensors associated with the bend;
based on the amount of strain determined for the bends of the plurality of struts, calculate a magnitude and a direction of the force;
graphically indicate on the display the magnitude and the direction of the force.

19. A catheter for measuring a force, the catheter comprising:
a proximal segment;
a distal segment;
a spring segment that extends from the proximal segment to the distal segment, the spring segment configured to permit relative movement between the distal segment and the proximal segment in response to application of the force on the distal segment, the spring segment comprising a plurality of struts, each strut extending from the proximal segment to the distal segment, each strut comprising a preformed curve that is bowed radially inward with respect to the catheter and each preformed curve is located between the distal segment and the proximal segment, wherein the plurality of struts are configured to:
mechanically support the distal segment in a base orientation with respect to the proximal segment,
flex at the preformed curves as the distal segment moves relative to the proximal segment in response to the application of the force, and
resiliently return the distal segment to the base orientation with respect to the proximal segment once the force has been removed; and
a plurality of sensors located on the bends of the struts and configured to output a plurality of signals indicative of dimensional changes in the curves of the struts.

20. A system for measuring a force, the system comprising:
a catheter comprising:
a proximal segment containing a proximal hub;
a distal segment containing a distal hub;
a spring segment that extends from the proximal segment to the distal segment, the spring segment configured to permit relative movement between the distal segment and the proximal segment in response to application of the force on the distal segment, the spring segment comprising a plurality of struts, each strut comprising a proximal end attached to the proximal hub, a distal end attached to the distal hub, and a preformed curve located between the distal end and the proximal end, each curve bowed radially inward with respect to the catheter, wherein the plurality of struts are configured to:
mechanically support the distal segment in a base orientation with respect to the proximal segment,
flex at the preformed curves when the distal segment moves relative to the proximal segment in response to the application of the force, and
resiliently return the distal segment to the base orientation with respect to the proximal segment once the force has been removed; and
a plurality of sensors mounted on the curves and configured to output a plurality of signals indicative of dimensional changes in the curves of the struts; and
control circuitry configured to:
receive the plurality of signals; and
calculate a magnitude and a direction of the force based on the plurality of signals.
